(19) United States
(12) Patent Application Publication
(10) Pub. No.: US 2025/0377291 A1
(43) Pub. Date: Dec. 11, 2025

(54) PHOTOACOUSTIC MEASUREMENT DEVICE AND PHOTOACOUSTIC MEASUREMENT METHOD

(71) Applicant: Hitachi High-Tech Corporation, Tokyo (JP)

(72) Inventors: Masuyoshi YAMADA, Tokyo (JP); Kosuke OHIRO, Tokyo (JP)

(21) Appl. No.: 19/230,421

(22) Filed: Jun. 6, 2025

(30) Foreign Application Priority Data
Jun. 11, 2024 (JP) 2024-094648

(51) Int. Cl. G01N 21/17; G01N 33/44

(52) U.S. Cl. CPC G01N 21/1702; G01N 33/442; G01N 2021/1706

(57) ABSTRACT

There is provided a photoacoustic measurement device including: a first acoustic cell configured to measure an external noise sound; a second acoustic cell configured to measure a reference sample by irradiating the reference sample with light of a first frequency modulated by a first frequency modulator; a third acoustic cell configured to measure an observation sample by irradiating the observation sample with light of a second frequency modulated by a second frequency modulator; and a processor configured to analyze acoustic wave data measured by the first acoustic cell, the second acoustic cell, and the third acoustic cell. The processor performs frequency analysis on the external noise sound, outputs a result obtained by setting, as the first frequency, a frequency having a low noise sound level among a result of the frequency analysis, modulating the first frequency with the first frequency modulator, measuring the reference sample with the second acoustic cell, analyzing a signal variation and a noise level variation of the reference sample to calculate a component determination threshold for the observation sample, and determining a component of the observation sample using the component determination threshold, causes the second frequency modulator to modulate the second frequency, which is different from the first frequency, and causes the third acoustic cell to measure the observation sample in parallel with the measurement of the reference sample, and changes, when the noise sound level of the second frequency is high in the result of the frequency analysis, the second frequency to the first frequency to measure the observation sample with the third acoustic cell.

### CLAIM OF PRIORITY

The present application claims priority from Japanese Patent application serial no. 2024-094648, filed on Jun. 11, 2024, the content of which is hereby incorporated by reference into this application.

### BACKGROUND OF THE INVENTION

### 1. Field of the Invention

The present invention relates to a configuration of a photoacoustic measurement device and a method thereof, and particularly to a technique that is effective when applied to measurement in an environment where an external noise sound is generated.

### 2. Description of Related Art

In recent years, there has been an increasing need for non-destructive inspection of organic substances such as plastic in recycling processes. When impurities are mixed into plastic or when components are altered due to aging, quality deterioration of a product after recycling is caused. Therefore, it is necessary to rapidly analyze components in the plastic. Photoacoustic spectroscopy is effective for non-destructive inspection of organic substances, as a measurement target can be measured regardless of a state (solid, liquid, gas, or powder) thereof.

A principle of a physical property measuring device using the photoacoustic spectroscopy is as follows. When a measurement target is irradiated with periodic intermittent light from a light source, the measurement target absorbs the light to generate heat, which is converted into kinetic energy that causes a surface to repeatedly expand and contract locally. As a result, pressure in a space produces sound waves (that is, photoacoustic waves), which are detected by a microphone. Basically, sound waves corresponding to a frequency of the intermittent light emitted from the light source are measured, and a detection result thereof is subjected to signal processing (physical property analysis), thereby enabling evaluation of substances and impurities.

However, there are various external environmental sounds in manufacturing sites such as a recycling plant, and photoacoustic waves generated by a photoacoustic effect are weak. Therefore, in order to perform non-destructive inspection with high sensitivity and high accurate, it is necessary to configure a measurement system so as to minimize the influence of the external noise sound.

As a background art of the present technical field, for example, there is a technique as disclosed in Patent Literature 1. Patent Literature 1 discloses a technique in which two photoacoustic cells are used, one cell is filled with a target gas for an odor measurement and the other cell is filled with odorless ambient air to measure an external ambient noise sound, and a measurement value of the latter is subtracted from a measurement value of the former to perform noise cancellation, thereby reducing an ambient environmental noise sound.

### CITATION LIST

### Patent Literature

Patent Literature 1: JP2021-179332A

### SUMMARY OF THE INVENTION

In the photoacoustic spectroscopy, it is preferable to perform frequency analysis of an external noise sound and shift a frequency of an intermittent light with which a photoacoustic cell is irradiated, that is, a frequency to be measured for a photoacoustic wave from a frequency at which an intensity of the external noise sound is strong. When a strong external noise sound is superimposed on the frequency to be measured and cannot be completely removed by noise cancellation, a large error occurs in a measurement result, resulting in poor accuracy of an inspection result.

Since an ambient external noise sound is generally not constant and changes over time, it is effective to monitor the external noise sound and check whether a strong noise sound is occurring at a frequency same as that of the intermittent light used in the photoacoustic cell. However, when the external noise sound becomes louder during measurement and the frequency used for the measurement is changed to another frequency at which an intensity of a noise sound is relatively low, sensitivity of the measurement before the change is changed, so that it is necessary to retake thresholds for sensitivity calibration and inspection determination, and an unknown sample to be measured may not be inspected during the work.

When the noise cancellation is performed using two cells according to the technique in Patent Literature 1, it may be difficult to completely cancel the external noise sound due to a machine difference between two microphones that measure a photoacoustic wave of each cell and a fact that positions of the two microphones are not completely the same with respect to a location where the noise sound is generated, and there is room for improvement.

Therefore, an object of the invention is to provide a photoacoustic measurement device and a photoacoustic measurement method that are less susceptible to an external noise sound and can continuously perform a highly accurate measurement according to a changing external environment.

In order to solve the above problems, the invention provides a photoacoustic measurement device including: a first acoustic cell configured to measure an external noise sound; a second acoustic cell configured to measure a reference sample by irradiating the reference sample with light of a first frequency modulated by a first frequency modulator; a third acoustic cell configured to measure an observation sample by irradiating the observation sample with light of a second frequency modulated by a second frequency modulator; and a processor configured to analyze acoustic wave data measured by the first acoustic cell, the second acoustic cell, and the third acoustic cell. The processor performs frequency analysis on the external noise sound, outputs a result obtained by setting, as the first frequency, a frequency having a low noise sound level among a result of the frequency analysis, modulating the first frequency with the first frequency modulator, measuring the reference sample with the second acoustic cell, analyzing a signal variation and a noise level variation of the reference sample to calculate a component determination threshold for the observation sample, and determining a component of the observation sample using the component determination threshold, causes the second frequency modulator to modulate the second frequency, which is different from the first frequency, and causes the third acoustic cell to measure the observation sample in parallel with the measurement of the reference sample, and changes, when the noise sound level of the second frequency is high in the result of the frequency analysis, the second frequency to the first frequency to measure the observation sample with the third acoustic cell.

The invention provides a photoacoustic measurement device including: a first acoustic cell configured to measure an external noise sound; a second acoustic cell different from the first acoustic cell; and a processor configured to analyze acoustic wave data measured by the first acoustic cell and the second acoustic cell. An observation sample is measured with the second acoustic cell using a second frequency. The processor performs frequency analysis on the external noise sound measured with the first acoustic cell, sets, as a first frequency, a frequency having a low noise sound level among a result of the frequency analysis, and changes, when a noise sound level of the second frequency is high in the result of the frequency analysis, the second frequency to the first frequency to measure the observation sample with the second acoustic cell.

The invention provides a photoacoustic measurement method including: (a) performing frequency analysis on an external noise sound; (b) outputting a result obtained by setting, as a first frequency, a frequency having a low noise sound level among a result of the frequency analysis in the step (a), modulating the first frequency with a first frequency modulator, measuring a reference sample with a second acoustic cell, analyzing a signal variation and a noise level variation of the reference sample to calculate a component determination threshold for an observation sample, and determining a component of the observation sample using the component determination threshold; (c) causing a second frequency modulator to modulate a second frequency, which is different from the first frequency, and causing a third acoustic cell to measure the observation sample in parallel with the measurement of the reference sample; and (d) changing, when a noise sound level of the second frequency is high in the result of the frequency analysis in the step (a), the second frequency to the first frequency to measure the observation sample with the third acoustic cell.

According to the invention, a photoacoustic measurement device and a photoacoustic measurement method that are less susceptible to an external noise sound and can continuously perform a highly accurate measurement according to a changing external environment can be implemented.

Accordingly, highly accurate non-destructive inspection using a photoacoustic effect can be enabled.

Problems, configurations, and effects other than those described above will be clarified by the following description of embodiments.

### BRIEF DESCRIPTION OF THE DRAWINGS

FIG. 1 is a diagram illustrating a schematic configuration of an inspection device according to Embodiment 1 of the invention.

FIG. 2 is a diagram illustrating details of a photoacoustic cell.

FIG. 3 is a diagram illustrating an example in which external noise is measured with a first photoacoustic cell.

FIG. 4 is a diagram illustrating an example in which a reference sample is measured with a second photoacoustic cell.

FIG. 5 is a diagram illustrating an example in which an observation sample is measured with a third photoacoustic cell.

FIG. 6 is a diagram illustrating an example in which the reference sample is measured with the second photoacoustic cell.

FIG. 7 is a flowchart illustrating a photoacoustic measurement method according to Embodiment 1 of the invention.

FIG. 8 is a diagram illustrating a relationship between a frequency of irradiation light and sensitivity and a fluctuation of a reference sample measurement peak in a photoacoustic measurement.

FIG. 9 is a diagram illustrating a schematic configuration of an inspection device according to Embodiment 3 of the invention.

FIG. 10 is a diagram illustrating a schematic configuration of an inspection device according to Embodiment 4 of the invention.

### DESCRIPTION OF EMBODIMENTS

Hereinafter, embodiments of the invention will be described with reference to the drawings. In the drawings, the same configurations are denoted by the same reference signs, and a detailed description of the repeating parts is omitted.

An inspection device of the present embodiment measures an external noise sound with a photoacoustic cell containing no sample (hereinafter, also simply referred to as an “acoustic cell” or a “cell”), measures a photoacoustic wave by putting a sample serving as an inspection reference, such as a known sample containing no impurities, in a reference sample measurement cell, and measures an observation sample to be inspected by putting the observation sample in an unknown sample measurement cell.

In the cell for measuring the external noise sound, it is confirmed whether an intensity of the external noise sound is increasing with respect to a frequency used for inspection. When the intensity of an external noise sound increases, another candidate frequency for switching, that is, a frequency at which the intensity of the external noise sound is low is selected and transmitted to the inspection device.

In the cell for measuring a reference sample, sensitivity, a noise level, and the like of the reference sample are measured using a frequency to be used for inspection, and a determination threshold used for analysis of a measurement result of the unknown sample measurement cell is obtained and transmitted to the inspection device. Further, the sensitivity and the noise level of the reference sample are measured using the other candidate frequency for switching that is obtained in the external noise sound measurement.

In the unknown sample measurement cell, a photoacoustic wave derived from the observation sample is measured, and a necessary inspection result is analyzed using the determination threshold received from the inspection device.

### Embodiment 1

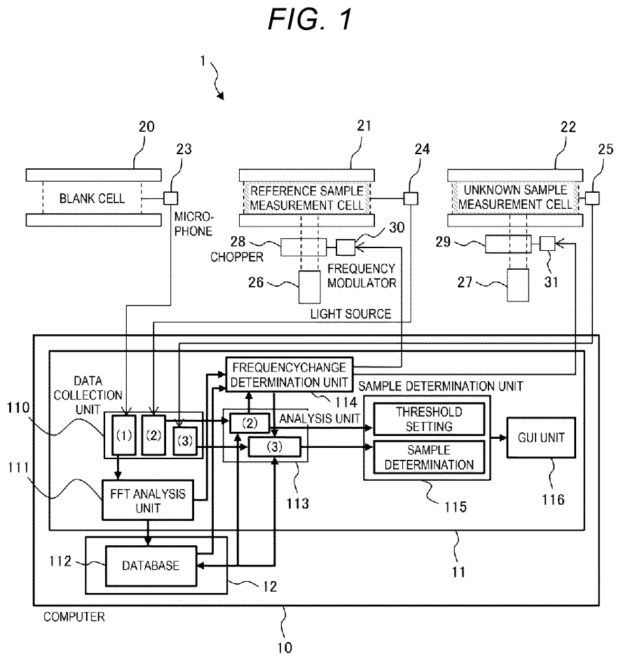

A photoacoustic measurement device and a photoacoustic measurement method according to Embodiment 1 of the invention will be described with reference to FIGS. 1 to 7.

FIG. 1 is a diagram illustrating a schematic configuration of an inspection device 1 of the present embodiment.

As illustrated in FIG. 1, the inspection device 1 of the present embodiment includes, as main components, a computer 10 and a plurality of (three in FIG. 1) acoustic cells 20, 21, and 22.

The acoustic cell 20 is an acoustic cell for measuring an external noise sound, and corresponds to a “first acoustic cell”. The acoustic cell 21 is an acoustic cell for measuring a sample serving as a reference, and corresponds to a “second acoustic cell”. The acoustic cell 22 is an acoustic cell for measuring an observation sample to be inspected by a user, and corresponds to a “third acoustic cell”. Microphones 23, 24, and 25 that measure sounds generated in the acoustic cells 20, 21, and 22 are electrically connected to the computer 10 in a wired or wireless manner.

The acoustic cell 21 and the acoustic cell 22 are provided with light sources 26 and 27 for irradiating an internal sample with light, optical path choppers or optical path shutters 28 and 29 for generating intermittent light, and frequency modulators 30 and 31 for controlling an intermittent light frequency. The frequency modulators 30 and 31 receive a set value of a frequency from the computer 10, and control the optical path choppers or optical path shutters 28 and 29 to emit intermittent light of the frequency.

The computer 10 includes a processor 11 and a memory 12. Further, the computer 10 may include a communication circuit, an input and output circuit, a user interface device, and the like (not illustrated).

The memory 12 is a storage device including a main storage device and an auxiliary storage device. A type of storage element used in the memory 12 is not limited. The memory 12 is provided with a database 112 having a function of accumulating data collected by a data collection unit 110.

The processor 11 is a circuit that performs calculation processing. The processor 11 includes, for example, a central processing unit (CPU) or a graphics processing unit (GPU), or a combination of these. The processor 11 is not limited to a CPU or a GPU, and another semiconductor device may be used as the processor 11 as long as it is a subject that executes predetermined processing.

The processor 11 includes the data collection unit 110, a fast Fourier transform (FFT) analysis unit 111, an analysis unit 113, a frequency change determination unit 114, a sample determination unit 115, and a GUI unit (a user interface unit) 116. In other words, the processor 11 executes a predetermined computer program stored in the memory 12 to implement processing of collecting data (110), processing of performing FFT analysis of data (111), processing of analyzing data (113), processing of determining a sample based on an analysis result (115), and processing of inputting and outputting data to and from a user interface device (116).

The data collection unit 110 has a function of collecting an acoustic cell output value (hereinafter, also simply referred to as an “output value” or a “measurement value”) from the acoustic cells 20, 21, and 22. The FFT analysis unit 111 has a function of performing frequency analysis on the output value obtained from the acoustic cell 20. As long as the FFT analysis unit 111 has a function of performing frequency analysis on the obtained acoustic data, a method thereof is not limited to FFT, and other methods such as sweeping a lock-in frequency of a substrate circuit may also be used. The data collection unit 110 measures data such as a relationship between a frequency and a signal intensity, a noise level, and a fluctuation in signal intensity in the reference sample measurement that are obtained so far in the acoustic cell 21 and the acoustic cell 22, and stores the data in the database 112. That is, the database 112 stores the data obtained from the data collection unit 110 and the FFT analysis unit 111 as a database.

The analysis unit 113 analyzes measurement results of the acoustic cell 21 and the acoustic cell 22 that are obtained by the data collection unit 110, obtains a relationship between an intermittent light frequency and a photoacoustic wave signal, acquires a noise level, and analyzes a signal intensity derived from a sample. The analysis unit 113 also sets a threshold necessary for the sample determination unit 115. The frequency change determination unit 114 determines whether to change the intermittent light frequency based on results of the FFT analysis unit 111 and the analysis unit 113, and selects a new frequency when the intermittent optical frequency is to be changed.

The sample determination unit 115 performs determination useful for a user based on a measurement result of an unknown sample from the analysis result obtained by the analysis unit 113. For example, when inspecting the presence or absence of impurities, an impurity concentration is calculated and ranked.

The GUI unit (the user interface unit) 116 includes an information providing device that provides information to a user and an information input device to which information is input by the user (both not illustrated). Examples of the information providing device include a monitor display, a printer, a voice synthesis device, and a lamp. Examples of the information input device include a keyboard switch, a touch panel, and a voice instruction device.

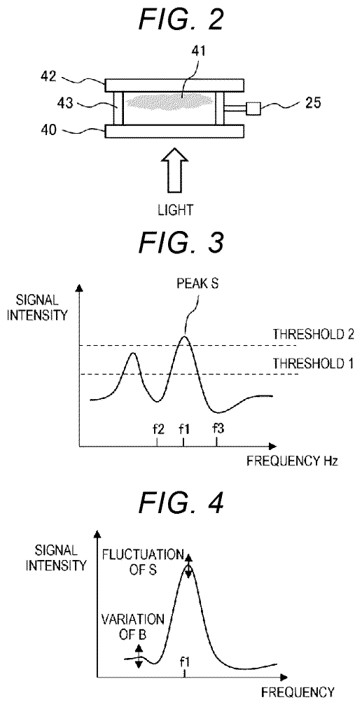

FIG. 2 is a diagram illustrating details of the photoacoustic cell.

The acoustic cells 20, 21, and 22 will be described with reference to FIG. 2. The acoustic cells 20, 21, and 22 are acoustic cells having basically the same dimensions and the same structure. The acoustic cell 22 will be described as an example.

The acoustic cell is formed of a space having a diameter of about several mm and a height of about several mm, and a glass window 40 for transmitting light into the cell is provided at one end. After a measurement target sample 41 such as a plastic piece is put in the cell, a cell space is sealed with a pressing plate 42 such as a metal plate. A component 43 at a portion to be irradiated with light is preferably made of a material such as metal that does not generate a photoacoustic wave. When the measurement target sample 41 is irradiated with intermittent light through the glass window 40, a photoacoustic wave is generated in the cell according to a sample component.

The frequency of the intermittent light will be described later, and as for a wavelength, it is common to use light in a mid-to-far infrared region of about several mm, and it is best to appropriately select a wavelength at which absorption of a measurement component is large and sensitivity of a photoacoustic wave is high. In order to ensure that light of an appropriate wavelength is directed to a sample, an optical filter (not illustrated) may be placed downstream of a light source, or a light source that emits a specific wavelength may be used. At this time, wavelengths of light used in the acoustic cell 21 and the acoustic cell 22 are basically the same. The microphone 25 for measuring a sound wave is connected to the space of the acoustic cell.

The acoustic cell 20 is an acoustic cell (a blank cell) for measuring an external noise sound. Therefore, a noise sound within the sealed space is measured by the microphone 23 without putting anything in the cell space. The measured noise sound is collected by the data collection unit 110, and a sound intensity distribution for each frequency can be analyzed by the FFT analysis unit 111. Note that a method other than FFT may be used as a method for performing frequency analysis.

The acoustic cell 21 is an acoustic cell for measuring a sample serving as a reference (a reference sample measurement cell). For example, when the inspection device 1 is used to measure the presence or absence of impurities in plastic, the sample serving as a reference is a pure plastic piece containing no impurities.

FIG. 4 is a diagram illustrating an example in which a reference sample is measured with the acoustic cell 21 (the second photoacoustic cell).

The reference sample in the acoustic cell 21 is irradiated with intermittent light of a frequency f1 generated by the light source 26, the frequency modulator 30, and the optical path chopper 28, and a photoacoustic wave is measured. When frequency analysis is performed on the obtained photoacoustic wave in the same manner as in the noise sound measurement with the acoustic cell 20, an intensity distribution having a peak S at the frequency f1 is obtained as illustrated in FIG. 4. At frequencies other than f1, a measurement noise level can be grasped. When the measurement is performed a plurality of times, a fluctuation in peak S and an average measurement noise level can be obtained.

In the acoustic cell 22, similar to the acoustic cell 21, light emitted from the light source 27 is turned into intermittent light of the frequency f1 by the frequency modulator 31 and the optical path chopper 29, and an observation sample in the acoustic cell 22 is irradiated with the intermittent light. A photoacoustic wave corresponding to a molecular structure and concentration of the observation sample is generated and detected by the microphone 25. After a measurement value obtained by the microphone 25 is collected by the data collection unit 110, the measurement value is analyzed by the analysis unit 113, and for example, the presence or absence of impurities is determined by the sample determination unit 115.

At this time, for example, a threshold for determining the presence or absence of impurities preferably changes according to the fluctuation in peak S or the change in measurement noise level that are obtained by the analysis of the acoustic cell 21 described above.

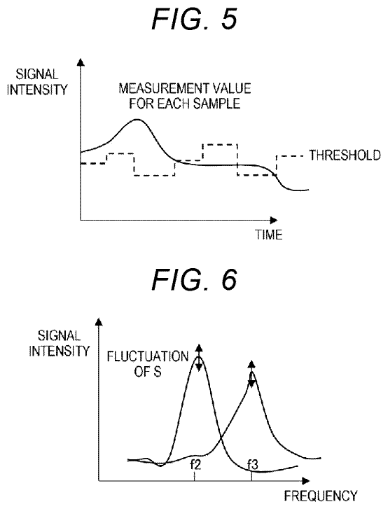

FIG. 5 is a diagram illustrating an example in which an observation sample is measured with the acoustic cell 22 (the third photoacoustic cell).

Since sensitivity of a target component in an observation sample to be measured with the acoustic cell 22 changes in an environment where various external noise sounds are present and change over time and in an environment where a temperature, a humidity, and the like affect detection sensitivity, a threshold changes according to the change as illustrated in FIG. 5. For example, when a measurement noise intensity measured with the acoustic cell 21 increases or when sensitivity increases, a determination threshold in the acoustic cell 22 also increases. When a fluctuation in peak S of the measurement noise intensity measured with the acoustic cell 21 increases, the determination threshold changes depending on whether a false positive or false negative rate is given more importance. Of course, in an environment where sensitivity and measurement noise do not vary significantly, the determination threshold may be fixed to perform the determination.

A flow during the normal measurement is as described above, and a flow for changing the frequency f1 used for the measurement to another frequency when an ambient external noise sound becomes louder will be described below.

FIG. 3 is a diagram illustrating an example in which external noise is measured with the acoustic cell 20 (the first photoacoustic cell).

As illustrated in FIG. 3, with respect to the obtained intensity distribution of noise sound for each frequency, a threshold for determining whether to change the frequency of the intermittent light to be used in the measurement is provided in advance. When the noise sound exceeds a threshold 2, a frequency change is executed, and when the noise sound exceeds a threshold 1, which is lower than the threshold 2, a preparation is made to switch to another frequency.

When the above-described preparation is started, first, a candidate frequency at which the intensity of the noise sound is low is picked up from a frequency analysis result of the noise sound of the acoustic cell 20. For example, in FIGS. 3, f2 and f3 at which a noise level is low with respect to the frequency f1 that is initially used are candidate frequencies. An example in which two candidate frequencies are picked up will be described below, but a single candidate frequency or three or more candidate frequencies may be picked up.

Using the picked-up candidate frequencies, the reference sample is measured with the acoustic cell 21 using intermittent light of the frequencies f2 and f3, respectively. Generally, a signal intensity of a photoacoustic wave increases as a frequency decreases, but since an experimental environment is also an influence, the signal intensity is actually measured with the acoustic cell 21 and the peak intensity S and the measurement noise level are obtained by the analysis unit 113. At this time, when the measurement of the observation sample with the acoustic cell 22 is continued at the frequency f1, an investigation for the frequency change and measurement of an unknown sample can be performed in parallel, and a measurement time loss can be eliminated. However, since the acoustic cell 21 cannot perform the measurement at the frequency f1 which is performed in the normal measurement, it is preferable to continuously use the value before start of the investigation as the threshold for determination in the acoustic cell 22.

FIG. 6 is a diagram illustrating an example in which the reference sample is measured with the acoustic cell 21 (the second photoacoustic cell).

As illustrated in FIG. 6, which frequency is to be selected is determined based on a measurement result at the two frequencies f2 and f3, that is, an investigation result. Examples of the method for this include a frequency at which measurement noise is lower, a frequency at which the peak intensity S is higher, and a frequency at which a temporal fluctuation in peak intensity S is smaller. Alternatively, there is also a method in which a ratio between a magnitude of the peak intensity S and a magnitude N of measurement noise is taken and a frequency at which a value thereof, i.e., an S/N ratio, is large is selected.

A new determination threshold to be used in the acoustic cell 22 is calculated at the frequency selected in this way, for example, f2. An investigation for a frequency change is completed here, and a value of the changed frequency f2 is transmitted to the frequency modulator 31 of the acoustic cell 22 to change the frequency of the intermittent light to f2. The threshold for determination is changed to the changed threshold described above.

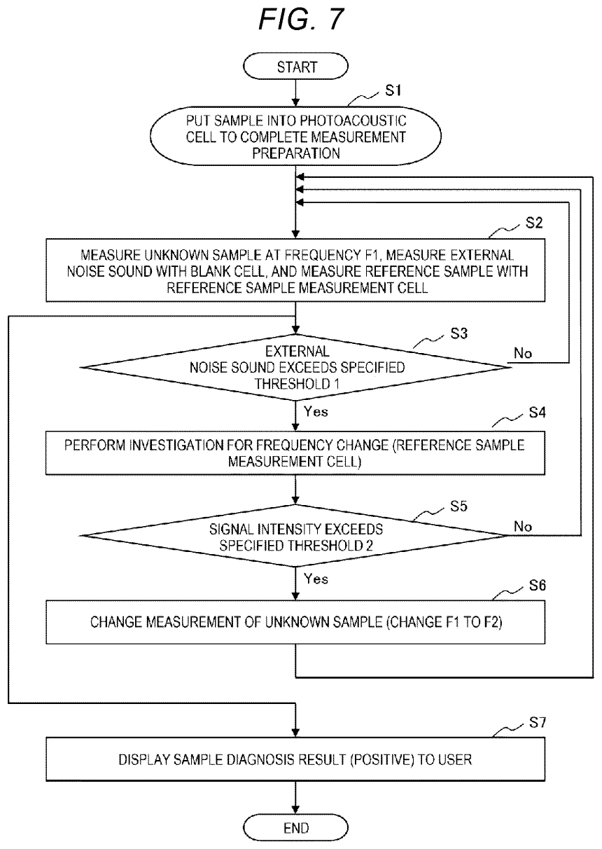

A series of measurement sequences described above is illustrated in FIG. 7. FIG. 7 is a flowchart illustrating a photoacoustic measurement method according to the present embodiment.

When the computer 10 starts processing, first, in step S1, a sample is put into a photoacoustic cell to complete measurement preparation.

Next, in step S2, an unknown sample is measured at the frequency f1. In parallel with this, an external noise sound is measured in a blank cell, and a reference sample is measured with a reference sample measurement cell.

Next, in step S3, it is determined whether the external noise sound exceeds the specified threshold 1. When it is determined that the external noise sound exceeds the specified threshold 1 (Yes), the processing proceeds to step S4. On the other hand, when it is determined that the external noise sound does not exceed the specified threshold 1 (No), the processing returns to step S2.

Next, in step S4, an investigation for a frequency change is performed in the reference sample measurement cell.

Next, in step S5, it is determined whether a signal intensity exceeds the specified threshold 2. When it is determined that the signal intensity exceeds the specified threshold 2 (Yes), the processing proceeds to step S6. On the other hand, when it is determined that the signal intensity does not exceed the specified threshold 2 (No), the processing returns to step S2.

Next, in step S6, a frequency for measuring the unknown sample is changed from f1 to f2, and the processing returns to step S2.

Finally, in step S7, a diagnosis result (positive) of the sample in step S2 is displayed to a user, and the processing ends.

By using such a device configuration and procedure, the following effects are obtained.

(1) A highly accurate measurement and determination can be made according to the changing external environment, and (2) measurement of an unknown observation sample can be continued even during an investigation for a frequency change, so that there is no impact on measurement throughput, i.e., no time loss.

In the configuration in FIG. 1, three acoustic cells are used, but a configuration using a plurality of cells rather than one acoustic cell for each function of the external noise sound measurement, the reference sample measurement, and the unknown observation sample may also be used.

### Embodiment 2

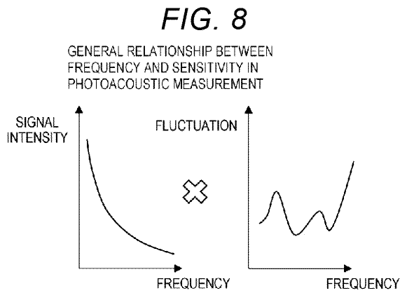

A photoacoustic measurement device and a photoacoustic measurement method according to Embodiment 2 of the invention will be described with reference to FIG. 8.

FIG. 8 is a diagram illustrating a relationship between a frequency of irradiation light and sensitivity and a fluctuation in reference sample measurement peak in a photoacoustic measurement.

The present embodiment is an example in which a result obtained by measuring a reference sample with the acoustic cell 21 in Embodiment 1 is stored in the database 112 and used as a reference when changing a frequency. That is, the present embodiment is an example in which the database 112 is used as an additional function to Embodiment 1.

As in Embodiment 1, the three acoustic cells 20, 21, and 22, the analysis unit 113, and the like are used. In Embodiment 1, during a normal measurement, a reference sample is measured with the acoustic cell 21 using the frequency f1 to be used in an unknown observation sample measurement with the acoustic cell 22, but it is different from Embodiment 1 in that a measurement result thereof and results obtained by measuring at other frequencies, for example, f2 and f3 in an investigation for a frequency change are stored in the database 112 and used for selecting a candidate frequency when changing a frequency.

The left diagram in FIG. 8 illustrates a relationship between an intermittent frequency and sensitivity (a signal intensity) of irradiation light in a photoacoustic measurement. Generally, sensitivity increases as a frequency decreases. On the other hand, at a measurement site, there are various undesired sounds and the like, which affect the measurement. For example, as illustrated in the right diagram in FIG. 8, an intermittent frequency and a fluctuation in reference sample measurement peak S or measurement noise are stored as a database from results of the normal measurement and measurement in preparation for a frequency change.

In Embodiment 1, in a process of selecting a candidate frequency to prepare for a frequency change of intermittent light when an external noise sound becomes louder, a candidate is selected only from a result of an external noise sound measurement in the acoustic cell 20, that is, a frequency at which an external noise sound is relatively small is selected, but in the present embodiment, in addition thereto, for example, a candidate frequency at which sensitivity is high and signal noise is low is selected from the stored database. At the selected frequency, as in Embodiment 1, an investigation measurement is performed again with the acoustic cell 21 to obtain a threshold determination value, or when a result is highly reproducible in the database, the frequency may be changed without performing a re-measurement.

An advantage of utilizing the database as in the present embodiment is that when performing the investigation for a frequency change, a stable frequency at which sensitivity is high is easily selected. Accordingly, when the unknown observation sample is measured with the acoustic cell 22 using the changed frequency, highly accurate determination can be made. Data for each frequency to be stored in the database may be investigated and accumulated with the acoustic cell 21 by changing the frequency finely and widely, but since this takes time, the already acquired data may be successively added and used to interpolate an insufficient portion.

### Embodiment 3

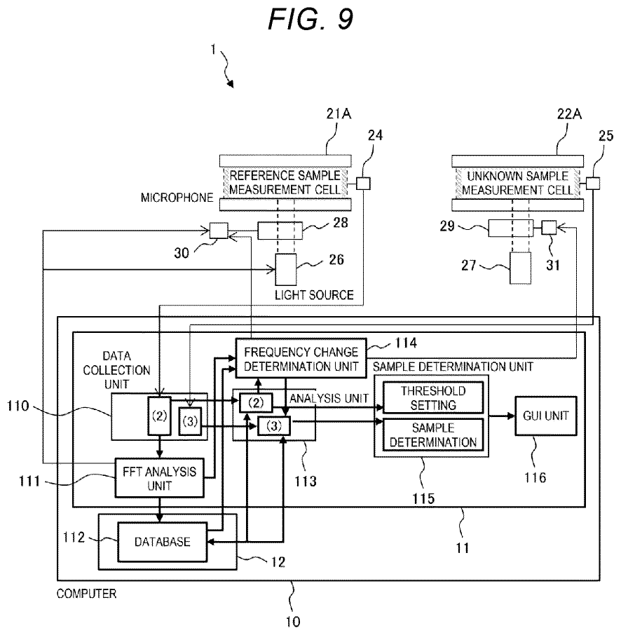

A photoacoustic measurement device and a photoacoustic measurement method according to Embodiment 3 of the invention will be described with reference to FIG. 9.

FIG. 9 is a diagram illustrating a schematic configuration of the inspection device 1 according to the present embodiment.

In Embodiment 1, three acoustic cells, i.e., the acoustic cells 20, 21, and 22 are used, whereas in the present embodiment, two acoustic cells, i.e., acoustic cells 21A and 22A are used. In the following embodiments including the present embodiment, differences from Embodiment 1 will be mainly described.

The acoustic cell 21A in the present embodiment has two functions, i.e., a function of the acoustic cell 20 and a function of the acoustic cell 21 in Embodiment 1. That is, ON/OFF of light with which a sample is irradiated is switched by sending a signal from the FFT analysis unit 111 to the light source 26 for performing control. A reference sample is put in the acoustic cell 21A, and when the light source 26 is turned OFF, no photoacoustic wave is generated from the reference sample and an external noise sound is measured. On the other hand, when the light source 26 is turned ON, the reference sample is irradiated with intermittent light to generate a photoacoustic wave. During an external noise sound measurement, a frequency of the frequency modulator 30 may be set to 0 while the light source 26 is kept ON, so that the sample is not irradiated with the intermittent light.

In Embodiment 1, the external noise sound measurement and the reference sample measurement are performed in different acoustic cells, but in the present embodiment, they are performed in the same acoustic cell 21A. A function of the acoustic cell 22A is the same as that of the acoustic cell 22 in Embodiment 1, and is used to measure an unknown observation sample and allow a user to make a necessary determination.

A measurement flow in the present embodiment is as follows.

First, the light source 26 of the acoustic cell 21A is turned OFF to measure an external noise sound. Thereafter, the light source 26 is turned ON to measure the reference sample. The obtained results are used for analysis of a determination threshold and the like as in Embodiment 1.

The light source 26 of the acoustic cell 21A is periodically switched ON and OFF to alternately perform the external noise sound measurement and the reference sample measurement. A frequency and timing of the measurement are determined appropriately depending on an environment where the measurement is performed.

Alternatively, the measurement flow may be such that the external noise sound measurement is performed with the light source 27 of the acoustic cell 22A turned OFF, and the reference sample measurement of the acoustic cell 21A is continued at all times. However, while the external noise sound is being measured with the acoustic cell 22A, an unknown observation sample cannot be measured.

By using the device configuration and the measurement flow as described above, there is an advantage that the number of acoustic cells required for measurement is reduced and the configuration is simplified. On the other hand, there is a disadvantage that the acoustic wave measurement of the reference sample or the observation sample cannot be performed at the same time while the light source is turned OFF.

### Embodiment 4

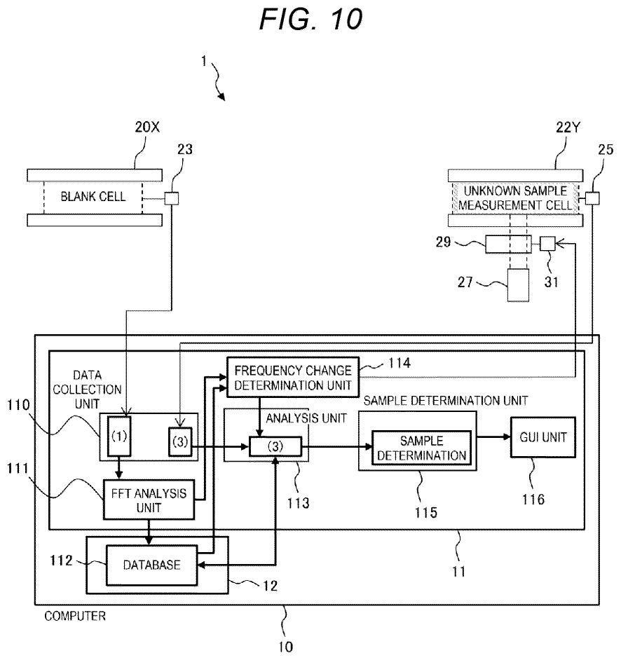

A photoacoustic measurement device and a photoacoustic measurement method according to Embodiment 4 of the invention will be described with reference to FIG. 10.

FIG. 10 is a diagram illustrating a schematic configuration of the inspection device 1 according to the present embodiment.

In Embodiment 1, three acoustic cells, i.e., the acoustic cells 20, 21, and 22 are used, whereas in the present embodiment, two acoustic cells, i.e., an acoustic cell 20X and an acoustic cell 22Y are used.

The acoustic cell 20X has a function, i.e., a function of the acoustic cell 20 in Embodiment 1. That is, an external noise sound is measured as in Embodiment 1. In the present embodiment, since there is no cell for measuring a reference sample as in Embodiment 1, the present embodiment is effective in a case of a measurement target without a sensitivity variation or a baseline fluctuation.

An unknown sample to be measured with the acoustic cell 22Y changes depending on a frequency of intermittent light with which the unknown sample is to be irradiated, and a relationship between a frequency and sensitivity as illustrated in FIG. 8 is collected in advance or made theoretically obtainable.

A measurement flow in the present embodiment is as follows.

First, an external noise sound is measured with the acoustic cell 20X. In the acoustic cell 22Y, the predetermined intermittent light frequency f1 is used to measure an unknown sample.

When a noise level of the intermittent light frequency to be used is high according to a measurement result of the acoustic cell 20X, the frequency is switched to another frequency. In the acoustic cell 22Y, the unknown sample is measured using the other intermittent light frequency, but a threshold for inspection determination is changed based on a relationship between a frequency and sensitivity obtained in advance to perform determination.

By using the device configuration and the measurement flow as described above, there is an advantage that the number of acoustic cells required for measurement is reduced and the configuration is simplified. On the other hand, there is a disadvantage that when a sensitivity variation or a baseline variation occurs due to a change in measurement environment, measurement accuracy deteriorates since a reference sample is not measured.

The invention is not limited to the embodiments described above, and includes various modifications. For example, the embodiments described above have been described in detail to facilitate understanding of the invention, and the invention is not necessarily limited to those including all the configurations described above. A part of a configuration of a certain embodiment can be replaced with a configuration of another embodiment, and the configuration of another embodiment can be added to a configuration of a certain embodiment. A part of a configuration of each embodiment may be added to, deleted from, or replaced with another configuration.

A part or all of the configurations, functions, processing units, processing methods, or the like described above may be implemented by hardware by, for example, designing with an integrated circuit. The above configurations, functions, and the like may be implemented by software by a processor interpreting and executing a program for implementing each function. Information such as a program, a table, and a file for implementing each function can be stored in a recording device such as a memory, a hard disk, and a solid state drive (SSD), or in a recording medium such as an IC card, an SD card, and a DVD.

### APPENDIX

The present invention also has the following features.

1. A photoacoustic measurement device comprising:
a first acoustic cell configured to measure an external noise sound;
a second acoustic cell configured to measure a reference sample by irradiating the reference sample with light at a first frequency modulated by a first frequency modulator;
a third acoustic cell configured to measure an observation sample by irradiating the observation sample with light at a second frequency modulated by a second frequency modulator; and
a processor configured to analyze acoustic wave data measured by the first acoustic cell, the second acoustic cell, and the third acoustic cell, in which
the processor
   performs frequency analysis on the external noise sound, and
   outputs a result obtained by setting, as the first frequency, a frequency having a low noise sound level among a result of the frequency analysis, modulating the first frequency with the first frequency modulator, measuring the reference sample with the second acoustic cell, analyzing signal variation and noise level variation of the reference sample to calculate a component determination threshold for the observation sample, and determining a component of the observation sample using the component determination threshold.

What is claimed is:

1. A photoacoustic measurement device comprising:
a first acoustic cell configured to measure an external noise sound;
a second acoustic cell configured to measure a reference sample by irradiating the reference sample with light of a first frequency modulated by a first frequency modulator;
a third acoustic cell configured to measure an observation sample by irradiating the observation sample with light of a second frequency modulated by a second frequency modulator; and
a processor configured to analyze acoustic wave data measured by the first acoustic cell, the second acoustic cell, and the third acoustic cell, wherein
the processor
performs frequency analysis on the external noise sound,
outputs a result obtained by setting, as the first frequency, a frequency having a low noise sound level among a result of the frequency analysis, modulating the first frequency with the first frequency modulator, measuring the reference sample with the second acoustic cell, analyzing a signal variation and a noise level variation of the reference sample to calculate a component determination threshold for the observation sample, and determining a component of the observation sample using the component determination threshold,
causes the second frequency modulator to modulate the second frequency, which is different from the first frequency, and causes the third acoustic cell to measure the observation sample in parallel with the measurement of the reference sample, and
changes, when the noise sound level of the second frequency is high in the result of the frequency analysis, the second frequency to the first frequency to measure the observation sample with the third acoustic cell.

2. The photoacoustic measurement device according to claim 1, wherein
a frequency to which the second frequency is changed when measuring the observation sample with the third acoustic cell is calculated based on a database storing a measurement result of the reference sample measured with the second acoustic cell.

3. The photoacoustic measurement device according to claim 1, wherein
a frequency to which the second frequency is changed when measuring the observation sample with the third acoustic cell is selected such that measurement noise is low, a signal intensity is high, and a variation is small in the measurement of the reference sample with the second acoustic cell.

4. The photoacoustic measurement device according to claim 1, wherein
a wavelength of light with which a sample is irradiated with the second acoustic cell and the third acoustic cell is the same and is changed according to the sample.

5. A photoacoustic measurement device comprising:
a first acoustic cell configured to measure an external noise sound;
a second acoustic cell different from the first acoustic cell; and
a processor configured to analyze acoustic wave data measured by the first acoustic cell and the second acoustic cell, wherein
an observation sample is measured with the second acoustic cell using a second frequency, and
the processor
performs frequency analysis on the external noise sound measured with the first acoustic cell,
sets, as a first frequency, a frequency having a low noise sound level among a result of the frequency analysis, and
changes, when a noise sound level of the second frequency is high in the result of the frequency analysis, the second frequency to the first frequency to measure the observation sample with the second acoustic cell.

6. The photoacoustic measurement device according to claim 5, wherein
the first acoustic cell measures a reference sample by irradiating the reference sample with light of the first frequency, and measures the external noise sound without irradiation with the light of the first frequency.

7. The photoacoustic measurement device according to claim 6, wherein
the first frequency to which the second frequency is changed when measuring the observation sample with the second acoustic cell is selected such that measurement noise is low, a signal intensity is high, and a variation is small in the measurement of the reference sample with the first acoustic cell.

8. The photoacoustic measurement device according to claim 5, wherein
the first acoustic cell is a blank cell containing no sample, and
the second acoustic cell is an unknown sample measurement cell for measuring an observation sample.

9. A photoacoustic measurement method comprising:
(a) performing frequency analysis on an external noise sound;
(b) outputting a result obtained by setting, as a first frequency, a frequency having a low noise sound level among a result of the frequency analysis in the step (a), modulating the first frequency with a first frequency modulator, measuring a reference sample with a second acoustic cell, analyzing a signal variation and a noise level variation of the reference sample to calculate a component determination threshold for an observation sample, and determining a component of the observation sample using the component determination threshold;
(c) causing a second frequency modulator to modulate a second frequency, which is different from the first frequency, and causing a third acoustic cell to measure the observation sample in parallel with the measurement of the reference sample; and
(d) changing, when a noise sound level of the second frequency is high in the result of the frequency analysis in the step (a), the second frequency to the first frequency to measure the observation sample with the third acoustic cell.
